(19) United States
(12) Patent Application Publication
(10) Pub. No.: US 2025/0019684 A1
(43) Pub. Date: Jan. 16, 2025

(54) METHOD FOR MAINTAINING LAMELLAR STRUCTURE OF CELL

(71) Applicant: LOCUS CELL CO,.LTD., New Taipei City (TW)

(72) Inventors: Hong-Nerng Ho, New Taipei City (TW); Heng-Yu Liu, New Taipei City (TW); Chen-Wei Lan, New Taipei City (TW); Chung-Wei Chang, New Taipei City (TW); Bo-He Chen, New Taipei City (TW); Chui Wei Wong, New Taipei City (TW); Chun-Lin Liu, New Taipei City (TW)

(21) Appl. No.: 18/764,793

(22) Filed: Jul. 5, 2024

(30) Foreign Application Priority Data
Jul. 13, 2023 (TW) 112126224

(51) Int. Cl. C12N 11/04; C12N 5/00

(52) U.S. Cl. CPC C12N 11/04; C12N 5/0012

(57) ABSTRACT

A method for maintaining lamellar structure of cell, configured to manufacture a lamellar cell product, comprises the following steps of: seeding a predetermined number of cells in a culture device containing a first culture medium and culturing the predetermined number of cells for a predetermined time to form a cell planar structure; placing a mold in the culture device to cover at least an outer portion of the cell planar structure; adding a gel into the culture device with the mold to envelop the cell planar structure; and separating the gel and the cell planar structure enveloped therein from the culture device and the mold to form the lamellar cell product.

### BACKGROUND OF THE INVENTION

### 1. Field of the Invention

The present invention relates to a method for maintaining lamellar structure of cell, and more particularly, to a method for manufacturing and maintaining a lamellar cell product that can be applied in the field of medical engineering.

### 2. Description of the Prior Art

In recent years, with the advancement of medical engineering technology, the application of regenerative medicine in the treatment of clinical diseases has become more widespread. Regenerative medicine is a medical technology that can repair damaged tissues and organs through the regenerative ability of cells. It has a wide range of applications and is expected to improve and treat previously difficult-to-treat diseases, such as diabetes, neurological diseases, cardiovascular diseases and cancers, through the combination of medical technologies such as tissue engineering and molecular biology. Currently, regenerative medicine is mainly applied to organ repair, immune cell therapy and stem cell therapy.

In regenerative medicine, the research and application of cell therapy have attracted more and more attention. Cell therapy is the process of culturing or processing human cells in vitro, and delivering the cells into the patient's body for treatment. In recent years, domestic and foreign researchers have developed of “lamellar cell” for cell therapy, the technique is now widely used in the repair of human organs and tissues, such as the heart, esophagus, joints, teeth and corneas.

However, it is known that during the process of culture preparation, the lamellar cell is prone to curling from the outer portion of the lamellar cell, so that the lamellar cell is unable to adhere to the cell culture dish continuously, and the lamellar cell will be detached from the cell culture dish by itself. In addition, lamellar cell is prone to deformation and damage due to sticking, adhesion, and crumpling during harvesting, transportation, and storage. Therefore, in the lamellar cell culture and preparation of the prior art, it is very likely to waste higher production costs and more labor time to repair the damage of the lamellar cell. Moreover, if the damage of the lamellar cell is too severe to be repaired, the lamellar cell can only be discarded, which leads to an increase in the cost of production of lamellar cell.

Therefore, it is necessary to develop a new technology that can avoid the curling or self-expulsion of the lamellar cell during the culture process and can harvest complete lamellar cell to solve the problems of the prior art.

### SUMMARY OF THE INVENTION

In view of this, one scope of the present invention is to provide a method for maintaining lamellar structure of cell, configured to manufacture a lamellar cell product. The method for maintaining lamellar structure of cell comprises the following steps of: seeding a predetermined number of cells in a culture device containing a first culture medium and culturing the predetermined number of cells for a predetermined time to form a cell planar structure; placing a mold in the culture device to cover at least an outer portion of the cell planar structure; adding a gel into the culture device with the mold to envelop the cell planar structure; and separating the gel and the cell planar structure enveloped therein from the culture device and the mold to form the lamellar cell product.

Wherein, the step of separating the gel and the cell planar structure enveloped therein from the culture device and the mold to form the lamellar cell product further comprises the following steps of: adding a second culture medium to separate the gel and the cell planar structure enveloped therein from the culture device; removing the mold to separate the gel and the cell planar structure enveloped therein from the mold to form the lamellar cell product.

Wherein, the predetermined number of cells is in a range from 1.0×10⁵ to 1.2×10⁶ cells/cm².

Wherein, the gel further comprises a hydrogel.

Wherein, the culture device is one of a round culture dish, a square culture dish, and a polygonal culture dish.

Wherein, a shape of the mold is corresponding to a shape of the culture device.

Wherein, the mold further comprises a first mold and a second mold.

Wherein, placing the mold in the culture device to cover at least the outer portion of the cell planar structure further comprises the following steps of: placing the first mold and the second mold in the culture device in sequence; wherein the first mold is disposed on the cell planar structure and covers at least an outer portion of the cell planar structure, and the second mold is disposed on the first mold.

Wherein, a material of the first mold is selected from glass, metal, and plastic; and a material of the second mold is selected from glass, metal, and plastic.

Wherein, the first mold is a ring-shaped structure, the ring-shaped structure is one of a circle ring, a square ring and a polygon ring.

In summary, the present invention provides a method for maintaining lamellar structure of cell, configured to manufacture a lamellar cell product. The present invention can prevent lamellar cell from curling and peeling off from the cell culture device by placing the mold in the culture device to envelop the cell planar structure during the culture process. In addition, the present invention can envelop the cell planar structure by adding the gel to the cell culture device with the mold, and prevent deformation and damage of the lamellar cell due to sticking, adherence, and crumpling of the lamellar cell during harvesting, transportation, and storage by curing the gel. Furthermore, the gel can also be configured to support the structure of the lamellar cell to maintain the structure of the lamellar cell so that the lamellar cell can be directly removed and harvested intact and uncurled.

### BRIEF DESCRIPTION OF THE APPENDED DRAWINGS

FIG. 1 is a flowchart illustrating a method for maintaining lamellar structure of cell according to an embodiment of the present invention.

FIG. 2 is a flowchart illustrating a method for maintaining lamellar structure of cell according to another embodiment of the present invention.

FIG. 3 is a flowchart illustrating a method for maintaining lamellar structure of cell according to another embodiment of the present invention.

### DETAILED DESCRIPTION OF THE INVENTION

For the sake of the advantages, spirits and features of the present invention can be understood more easily and clearly, the detailed descriptions and discussions will be made later by way of the embodiments and with reference of the diagrams. It is worth noting that these embodiments are merely representative embodiments of the present invention, wherein the specific methods, devices, conditions, materials and the like are not limited to the embodiments of the present invention or corresponding embodiments. Moreover, the devices in the figures are only used to express their corresponding positions and are not drawing according to their actual proportion.

In the description of this specification, the description with reference to the terms “an embodiment”, “another embodiment” or “part of an embodiment” means that a particular feature, structure, material or characteristic described in connection with the embodiment including in at least one embodiment of the present invention. In this specification, the schematic representations of the above terms do not necessarily refer to the same embodiment. Furthermore, the particular features, structures, materials or characteristics described may be combined in any suitable manner in one or more embodiments. Furthermore, the indefinite articles “a” and “an” preceding a device or element of the present invention are not limiting on the quantitative requirement (the number of occurrences) of the device or element. Thus, “a” should be read to include one or at least one, and a device or element in the singular also includes the plural unless the number clearly refers to the singular.

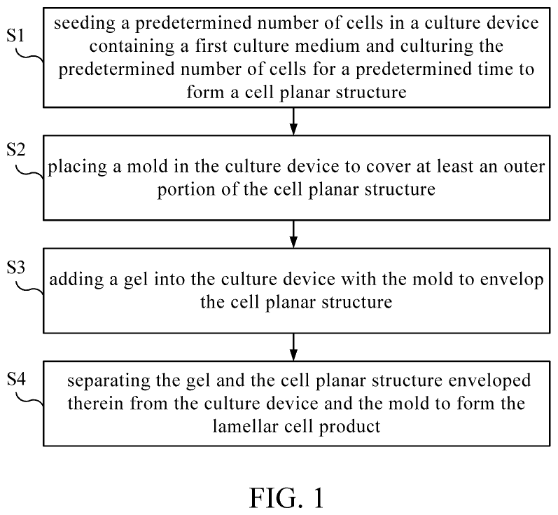

Please refer to FIG. 1, FIG. 1 is a flowchart illustrating a method for maintaining lamellar structure of cell according to an embodiment of the present invention. As shown in FIG. 1, in the present embodiment, the method for maintaining lamellar structure of cell comprises the following steps of: step S1: seeding a predetermined number of cells in a culture device containing a first culture medium and culturing the predetermined number of cells for a predetermined time to form a cell planar structure; step S2: placing a mold in the culture device to cover at least an outer portion of the cell planar structure; step S3: adding a gel into the culture device with the mold to envelop the cell planar structure; and step S4: separating the gel and the cell planar structure enveloped therein from the culture device and the mold to form the lamellar cell product.

In another embodiment, the method for maintaining lamellar structure of cell further comprises the following steps. Wherein, one step is performed after step S2: separating the cell planar structure covered by the mold from the culture device, and then step S3 is performed. Another step is performed after step S3: curing the gel enveloping the cell planar structure, and then step S4 is performed. In practice, the gel can be fixed by physical and chemical methods, such as UV irradiation, light source irradiation with special wavelength, heating, addition of chemical additives. The fixation method can be determined according to the physical and chemical properties of the gel selected by the user.

The present embodiment can be configured to manufacture a lamellar cell product, such as a somatic lamellar cell and a stem lamellar cell. In practice, the selection of the cells can be according to the user's needs to choose other cells that can form the lamellar cell. In step S1 of the present embodiment, the predetermined number of cells is in a range from 1.0×10⁵ to 1.2×10⁶ cells/cm². In practice, the range of the predetermined number of cells to be cultured can be determined according to the suitable growth density of different cell types. In addition, in this embodiment, the predetermined time for cell culture can be adjusted according to the cell growth conditions.

In step S2 of the present embodiment, the cell planar structure can be covered by the mold, and the mold is placed to cover the outer portion of the cell planar structure to avoid the lamellar cell from curling from the outer portion during the cell culture process, so as to prevent the lamellar cell from peeling off from the cell culture device. In this embodiment, the culture device can be a round culture dish, a square culture dish, or a polygonal culture dish. In practice, other devices or containers suitable for cell culture can also be selected, and the choice of the culture device can be further according to the characteristics of the cells. In addition, in the present embodiment, the mold can be a ring-shaped structure. The ring-shaped structure of the mold in the present embodiment covers the outer portion of the cell planar structure and presses the outer portion of the cell planar structure by the weight of the mold to prevent the outer portion of the planar structure of the cell from starting to curl during the process of separating the lamellar cell from the culture device and the mold, so as to prevent the lamellar cell from separating from the cell culture device and curling by itself. Moreover, the shape of the mold used in the present embodiment can be selected to correspond to the shape of the culture device, such as a round shape, a square shape, a polygonal shape. However, in practice, it is not limited to the aforementioned. Other molds that can cover at least the outer portion of the cell planar structure and correspond to the shape of the cell planar structure can also be selected according to the needs of the user.

In step S3 of the present embodiment, the gel can be added to support and maintain the structure of the lamellar cell, and the lamellar cell can be directly removed and harvested intact and uncurled from the culture device. In addition, in the present embodiment, the gel further comprises a hydrogel and can be a gelatin. In practice, the hydrogel is mainly composed of proteins extracted from the extracellular matrix (ECM) and have similar or identical molecular structure and composition to living organisms. Therefore, the hydrogel is highly biocompatible and less likely to cause inflammation. Because of the fluidic characteristic of the gel in the present embodiment, it can avoid the mold and pass through the gap between the mold and the cell planar structure, so the gel can enter and envelope the cell planar structure. In the present embodiment, the hydrogel is used to support the structure of the cell planar structure, but in practice, it is not limited to the aforementioned, other gels and materials with the same characteristics can be used in cell culture according to the user's needs. In addition, the gel in the present embodiment is added to the culture device with the mold to envelope the cell planar structure and maintain the integrity of the cell planar structure, so as to facilitate the harvesting of intact and non-curled lamellar cell. At the same time, the curing gel can prevent deformation and breakage of the lamellar cell due to sticking, adherence and crumpling of lamellar cell during harvesting, transportation and storage.

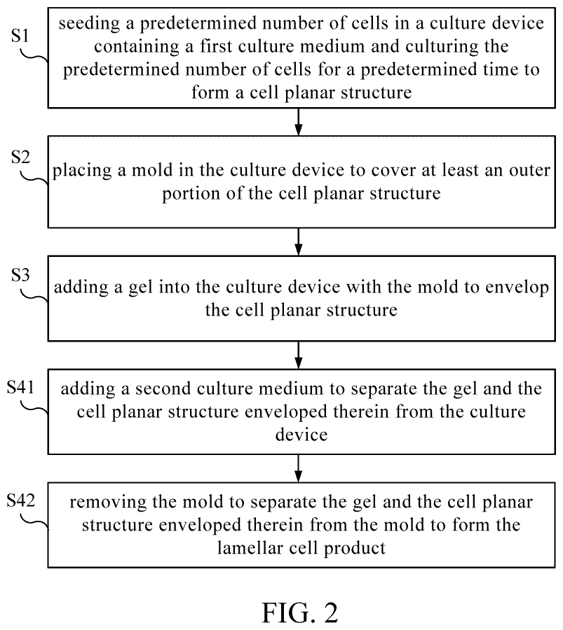

In addition to the embodiments described above, the present invention provides another embodiment of maintaining lamellar structure of cell. Please refer to FIG. 2. FIG. 2 is a flowchart illustrating a method for maintaining lamellar structure of cell according to another embodiment of the present invention. As shown in FIG. 2, the present embodiment differs from the aforementioned embodiment in that the method for maintaining lamellar structure of cell of the present embodiment further comprises step S41 and step S42, which are sequentially performed after step S3. Step S41: adding a second culture medium to separate the gel and the cell planar structure enveloped therein from the culture device; and Step S42: removing the mold to separate the gel and the cell planar structure enveloped therein from the mold to form the lamellar cell product. In the present embodiment, the first culture medium is configured to provide the nutrients necessary for cell growth; and the second culture medium is configured to separate the gel and the cell planar structure enveloped therein from the culture device, so as to facilitate the harvesting of the lamellar cell. At the same time, the second culture medium can provide the nutrients necessary for the lamellar cell. In practice, the first culture medium and the second culture medium can be selected according to the user's needs to be suitable for cell culture, and the first culture medium and the second culture medium can be selected with the same or different ingredients. It should be noted that the method for maintaining lamellar structure of cell of the present embodiment has substantially the same steps as the corresponding steps of the aforementioned embodiment, so it will not be described again herein.

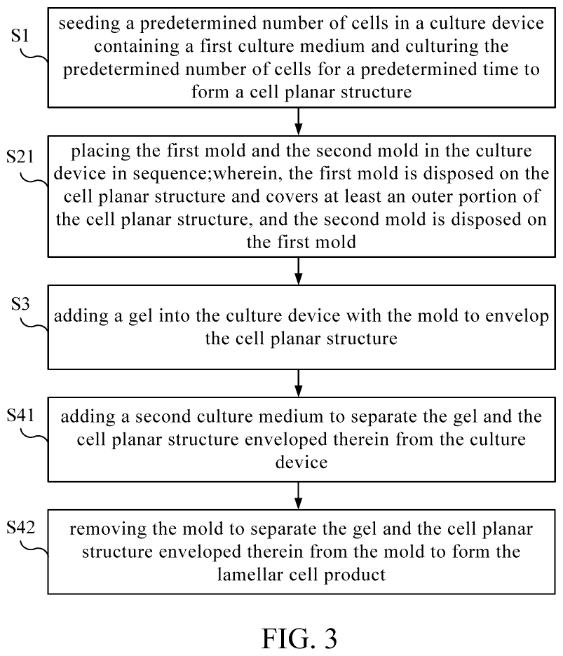

In practice, the mold in the present embodiment can further comprise a first mold and a second mold. The first mold and the second mold can be applied to the method for maintaining lamellar structure of cell of the present invention. Please refer to FIG. 3, FIG. 3 is a flowchart illustrating a method for maintaining lamellar structure of cell according to another embodiment of the present invention. As shown in FIG. 3, the present embodiment differs from the aforementioned embodiments in that the method for maintaining lamellar structure of cell in the present embodiment further comprises step S21, which is performed after step S1. Step S21: placing the first mold and the second mold in the culture device in sequence. In the present embodiment, the first mold is placed directly on the cell planar structure and covers at least an outer portion of the cell planar structure, so the cell planar structure can be fixed in the culture device. The second mold is placed on the first mold to provide weight, and the first mold can be fixed on the cell planar structure to avoid the curling of the lamellar cell and peeling off from the cell culture device by itself. In addition, the material of the first mold can be selected from glass, metal, and plastic; and the material of the second mold can be selected from glass, metal, and plastic. The material of the first mold and the second mold can be the same or different. However, in practice, this is not limited to the aforementioned, and other materials can be used to fix the cells and provide weight according to the user's needs. It should be noted that the method for maintaining lamellar structure of cell of the present embodiment has substantially the same steps as the corresponding steps of the aforementioned embodiment, so it will not be described again herein.

In the present embodiment, the first mold can be a ring-shaped structure, the ring-shaped structure is one of a circle ring, a square ring and a polygon ring. Since the cell planar structure is prone to curl and peel off from the cell culture device during culture, the outer portion of the cell planar structure is the most prone to start curling. Therefore, in the present embodiment, the first mold is placed on the outer portion of the cell planar structure and the second mold is placed on the first mold to fix the outer portion of the cell planar structure, so as to prevent the lamellar cell from curling from the outer portion and peeling off from the cell culture device. On the other hand, when the cell planar structure is exposed to external pressure, it can cause damage to parts of the cells. Therefore, in another embodiment, the first mold and the second mold can both be ring-shaped structures. Wherein, the first mold can be a lighter-weight plastic ring with a thinner ring wall, and the second mold can be a heavier-weight metal ring with a thicker ring wall. With the thinner and lighter ring of the first mold, the area of direct contact between the first mold and the cell planar structure is smaller when the first mold is placed on the outer portion of the cell planar structure, so the areas of damage to the cells and the number of cells are lower. At the same time, the second mold with the thicker and heavier ring is placed on the first mold to provide the weight to the first mold for fixing the first mold to the cell planar structure. It should be noted that the method for maintaining lamellar structure of cell of the present embodiment has substantially the same steps as the corresponding steps of the aforementioned embodiment, so it will not be described again herein. In addition, in practice, the shape of the ring structure is not limited to the aforementioned, and the shape of the ring structure can be a triangular ring, an elliptical ring or other shapes according to the user's needs.

In summary, the present invention provides a method for maintaining lamellar structure of cell, configured to manufacture a lamellar cell product. The present invention can prevent lamellar cell from curling and peeling off from the cell culture device by placing the mold in the culture device to envelop the cell planar structure during the culture process. In addition, the present invention can envelop the cell planar structure by adding the gel to the cell culture device with the mold, and prevent deformation and damage of the lamellar cell due to sticking, adherence, and crumpling of the lamellar cell during harvesting, transportation, and storage by curing the gel. Furthermore, the gel can also be configured to support the structure of the lamellar cell to maintain the structure of the lamellar cell so that the lamellar cell can be directly removed and harvested intact and uncurled.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A method for maintaining lamellar structure of cell, configured to manufacture a lamellar cell product, the method comprising the following steps of:
seeding a predetermined number of cells in a culture device containing a first culture medium and culturing the predetermined number of cells for a predetermined time to form a cell planar structure;
placing a mold in the culture device to cover at least an outer portion of the cell planar structure;
adding a gel into the culture device with the mold to envelop the cell planar structure; and
separating the gel and the cell planar structure enveloped therein from the culture device and the mold to form the lamellar cell product.

2. The method for maintaining lamellar structure of cell of claim 1, wherein the step of separating the gel and the cell planar structure enveloped therein from the culture device and the mold to form the lamellar cell product further comprises the following steps of:
adding a second culture medium to separate the gel and the cell planar structure enveloped therein from the culture device;
removing the mold to separate the gel and the cell planar structure enveloped therein from the mold to form the lamellar cell product.

3. The method for maintaining lamellar structure of cell of claim 1, wherein the predetermined number of cells is in a range from 1.0×10⁵ to 1.2×10⁶ cells/cm².

4. The method for maintaining lamellar structure of cell of claim 1, wherein the gel further comprises a hydrogel.

5. The method for maintaining lamellar structure of cell of claim 1, wherein the culture device is one of a round culture dish, a square culture dish, and a polygonal culture dish.

6. The method for maintaining lamellar structure of cell of claim 1, wherein a shape of the mold is corresponding to a shape of the culture device.

7. The method for maintaining lamellar structure of cell of claim 1, wherein the mold further comprises a first mold and a second mold.

8. The method for maintaining lamellar structure of cell of claim 7, wherein placing the mold in the culture device to cover at least the outer portion of the cell planar structure further comprises the following steps of:
placing the first mold and the second mold in the culture device in sequence;
wherein, the first mold is disposed on the cell planar structure and covers at least an outer portion of the cell planar structure, and the second mold is disposed on the first mold.

9. The method for maintaining lamellar structure of cell of claim 7, wherein a material of the first mold is selected from glass, metal, and plastic; and a material of the second mold is selected from glass, metal, and plastic.

10. The method for maintaining lamellar structure of cell of claim 7, wherein the first mold is a ring-shaped structure, the ring-shaped structure is one of a circle ring, a square ring and a polygon ring.
